(19) United States
(12) Patent Application Publication
(10) Pub. No.: US 2015/0374398 A1
(43) Pub. Date: Dec. 31, 2015

(54) LEAD EXTRACTION

(71) Applicant: LEADR MEDICAL LTD, Tel Aviv (IL)

(72) Inventor: Idan TOBIS, Beth Hashmonai (IL)

(73) Assignee: LEADR MEDICAL LTD, Tel Aviv (IL)

(21) Appl. No.: 14/316,025

(22) Filed: Jun. 26, 2014

(51) Int. Cl. A61B 17/32

(52) U.S. Cl. CPC A61B 17/32002; A61B 2017/00243

(57) ABSTRACT

Apparatus for removing a lead from a lumen of a subject including a flexible control tube shaped to define a control-tube lumen; and a flexible grasping tube shaped to define a grasping-tube lumen. The grasping tube is disposed within the control-tube lumen and includes a torque-transfer non-distal portion; and a helically-cut distal portion, that is (a) fixed at a distal end thereof to a distal end of the control tube, and (b) rotatable at least at a proximal end thereof with respect to the control tube. The helically-cut distal portion is configured to grasp the lead within the grasping-tube lumen upon the proximal end of the helically-cut distal portion being rotated with respect to the control tube.

### FIELD OF THE INVENTION

The present invention relates to apparatus and methods for extracting a lead, such as a cardiac pacemaker lead, from a subject.

### BACKGROUND

In some situations, an implanted lead, such as a cardiac pacemaker lead, may stop working, or an infection may set in at the implant site. In such situations, it may be desired to extract the lead from the subject.

### SUMMARY OF THE INVENTION

Applications of the present invention provide apparatus for removing a lead (e.g., a cardiac pacemaker lead) from a lumen (e.g., a blood-vessel lumen) of a subject. The apparatus includes at least two tubes: a flexible control tube, and a flexible grasping tube disposed within a lumen of the control tube. The flexible grasping tube includes a torque-transfer non-distal portion and a helically-cut distal portion. The helically-cut distal portion is fixed at a distal end thereof to a distal end of the control tube, and is rotatable at least at a proximal end thereof with respect to the control tube. When the proximal end of the helically-cut distal portion is rotated with respect to the control tube, the helically-cut distal portion grasps the lead within a lumen of the grasping tube. The rotation of the proximal end of the helically-cut distal portion may be effected via the application of torque to the proximal end of the grasping tube, the torque-transfer non-distal portion transferring the applied torque to the proximal end of the helically-cut distal portion.

There is therefore provided, in accordance with some applications of the present invention, apparatus for removing a lead from a lumen of a subject, the apparatus including:

a flexible control tube shaped to define a control-tube lumen; and

a flexible grasping tube shaped to define a grasping-tube lumen, the grasping tube being disposed within the control-tube lumen and including:
a torque-transfer non-distal portion; and
a helically-cut distal portion, that is (a) fixed at a distal end thereof to a distal end of the control tube, and (b) rotatable at least at a proximal end thereof with respect to the control tube,
the helically-cut distal portion being configured to grasp the lead within the grasping-tube lumen upon the proximal end of the helically-cut distal portion being rotated with respect to the control tube.

In some applications, the grasping tube is cylindrical, and a diameter of the torque-transfer non-distal portion of the grasping tube is between 2 and 6 mm.

In some applications, the control tube is cylindrical, and an inner diameter of the control tube is between 0.01 and 0.1 mm larger than an outer diameter of the grasping tube.

In some applications, a length of the helically-cut distal portion along a longitudinal axis of the helically-cut distal portion, in the absence of any external force or torque applied to the helically-cut distal portion, is between 5 and 30 mm.

In some applications, the grasping tube is cylindrical, and a diameter of the helically-cut distal portion, in the absence of any external force or torque applied to the helically-cut distal portion, is between 2 and 6 mm.

In some applications, the apparatus further includes, at a distal end of the control tube, a coring tool including one or more blades shaped to define respective cutting edges.

In some applications, the cutting edges face toward a central longitudinal axis of the control tube.

In some applications, the one or more blades include a plurality of blades disposed around a central longitudinal axis of the control tube.

In some applications, the control tube has a control tube inner diameter, and the cutting edges are within 0.1 and 0.3 mm of an inside wall of a hypothetical cylinder having an inner diameter equal to the control tube inner diameter and extending from the distal end of the control tube to a point that is distal to the coring tool.

In some applications, the apparatus further includes:

a flexible outer tube shaped to define an outer-tube lumen, the control tube being a middle tube shaped to fit inside the outer-tube lumen; and

a coring tool including one or more blades and disposed at a distal end of the outer tube.

In some applications, the torque-transfer non-distal portion of the grasping tube includes a laser-cut torque-transfer non-distal portion of the grasping tube.

In some applications, the laser-cut torque-transfer non-distal portion of the grasping tube includes a self-interlocking helical ribbon having a circumference that does not change upon the ribbon being rotated with respect to the control tube.

In some applications,

the helically-cut distal portion of the grasping tube is a first helically-cut distal portion of the grasping tube that is configured to grasp the lead within the grasping-tube lumen upon a proximal end of the first helically-cut distal portion being rotated with respect to the control tube in a first direction, and

the grasping tube further includes a second helically-cut distal portion that is proximal to the first helically-cut distal portion, the second helically-cut distal portion being configured to grasp the lead within the grasping-tube lumen upon a proximal end of the second helically-cut distal portion being rotated with respect to the control tube in a second direction that is opposite from the first direction.

There is further provided, in accordance with some applications of the present invention, apparatus for removing a lead from a lumen of a subject, the apparatus including:

a flexible grasping tube shaped to define a tube lumen, the grasping tube including a distal braided portion configured to grasp the lead within the tube lumen upon being longitudinally stretched; and

longitudinal control elements that include:
a first longitudinal control element extending from a proximal end of the apparatus to a distal end of the distal braided portion; and
a second longitudinal control element extending from the proximal end of the apparatus to a proximal end of the distal braided portion,

the first and second longitudinal control elements including:
respective flexible tubes shaped to define respective flexible-tube lumens thereof; and
respective longitudinal elements shaped to fit inside the flexible-tube lumens.

In some applications, the longitudinal elements are wires having respective diameters between 0.1 and 1 mm.

In some applications, respective inner diameters of the flexible tubes are between 0.2 and 2 mm greater than respective diameters of the longitudinal elements.

In some applications, the apparatus further includes a catheter shaped to define a catheter lumen, and the grasping tube is shaped to fit inside the catheter lumen.

In some applications, an inner diameter of the catheter is between 0.05 and 1 mm greater than an outer diameter of the grasping tube.

In some applications,

the distal braided portion is a first distal braided portion,

the grasping tube further includes a second distal braided portion configured to grasp the lead within the tube lumen upon being longitudinally stretched, the second distal braided portion being (a) proximal to the first distal braided portion, and (b) separated from the first distal braided portion by a non-braided portion of the grasping tube, and

the longitudinal control elements further include a third longitudinal control element extending from the proximal end of the apparatus to a proximal end of the second distal braided portion.

In some applications, the apparatus further includes a coring tool including one or more blades and disposed at a distal end of the grasping tube.

In some applications, the apparatus further includes:

a flexible control tube shaped to define a control-tube lumen, the grasping tube shaped to fit inside the control-tube lumen; and

a coring tool including one or more blades and disposed at a distal end of the control tube.

There is further provided, in accordance with some applications of the present invention, a method for removing a lead from a lumen of a subject, the method including:

inserting apparatus into the lumen, the apparatus including a grasping tube;

advancing a distal portion of the grasping tube to a distal portion of the lead;

grasping the lead within a grasping portion of the distal portion of the grasping tube, by reducing a diameter of the grasping portion; and

while grasping the lead, removing the lead.

In some applications, reducing the diameter of the grasping portion includes reducing the diameter of the grasping portion by rotating a proximal end of the grasping tube.

In some applications, reducing the diameter of the grasping portion includes reducing the diameter of the grasping portion by rotating a proximal end of a control tube, inside of which the grasping tube is disposed.

In some applications, reducing the diameter of the grasping portion includes longitudinally stretching the grasping portion.

In some applications, longitudinally stretching the grasping portion includes longitudinally stretching the grasping portion by pulling a longitudinal control element that extends from a proximal end of the apparatus to a proximal end of the grasping portion.

In some applications, longitudinally stretching the grasping portion includes longitudinally stretching the grasping portion by pushing a longitudinal control element that extends from a proximal end of the apparatus to a distal end of the grasping portion.

In some applications, the method further includes, while advancing the distal portion of the grasping tube to the distal portion of the lead, separating the lead from tissue of the subject by using a coring tool disposed at a distal end of the apparatus to core the tissue.

In some applications, advancing the distal portion of the grasping tube to the distal portion of the lead includes repeatedly executing a sequence of steps that includes:

at a site adjacent to a first tissue-to-lead adhesion site, grasping the lead within the grasping portion, by reducing a diameter of the grasping portion,

while grasping the lead within the grasping portion, using the coring tool to separate the lead from tissue of the subject at the first tissue-to-lead adhesion site, and

following the separating of the lead from the tissue at the first tissue-to-lead adhesion site, advancing the distal portion of the grasping tube to a site adjacent to a second tissue-to-lead adhesion site.

In some applications,

the distal portion of the grasping tube includes (a) a first grasping portion, and (b) a second grasping portion that is proximal to the first grasping portion, and

advancing the distal portion of the grasping tube to the distal portion of the lead includes repeatedly executing a sequence of steps that includes:
grasping the lead within the first grasping portion, by reducing a diameter of the first grasping portion,
while grasping the lead within the first grasping portion, advancing a proximal end of the second grasping portion,
following the advancing of the proximal end, grasping the lead within the second grasping portion, by reducing a diameter of the second grasping portion, and
while grasping the lead within the second grasping portion, advancing a distal end of the grasping tube.

In some applications, the method further includes, while grasping the lead within the second grasping portion, separating the lead from tissue of the subject by using a coring tool disposed at a distal end of the apparatus to core the tissue.

In some applications,

reducing the diameter of the first grasping portion includes reducing the diameter of the first grasping portion by rotating a proximal end of the grasping tube in a first direction, and

reducing the diameter of the second grasping portion includes rotating the proximal end of the grasping tube in a second direction that is opposite the first direction.

In some applications,

reducing the diameter of the first grasping portion includes reducing the diameter of the first grasping portion by rotating a proximal end of a control tube, inside of which the grasping tube is disposed, in a first direction, and

reducing the diameter of the second grasping portion includes reducing the diameter of the second grasping portion by rotating the proximal end of the control tube in a second direction that is opposite the first direction.

In some applications, reducing the diameter of the second grasping portion includes reducing the diameter of the second grasping portion by pushing a longitudinal control element that extends from a proximal end of the apparatus to a distal end of the second grasping portion.

In some applications, reducing the diameter of the first grasping portion includes reducing the diameter of the first grasping portion by pushing a longitudinal control element that extends from a proximal end of the apparatus to a distal end of the first grasping portion.

In some applications, reducing the diameter of the first grasping portion includes reducing the diameter of the first grasping portion by pulling a longitudinal control element that extends from a proximal end of the apparatus to a proximal end of the first grasping portion.

The present invention will be more fully understood from the following detailed description of applications thereof, taken together with the drawings, in which:

### BRIEF DESCRIPTION OF THE DRAWINGS

FIG. 1 is a schematic illustration of a method for removing a lead from a lumen of a subject, in accordance with some applications of the present invention;

FIGS. 2A-B are schematic illustrations of apparatus in a disassembled state, in accordance with some applications of the present invention;

FIG. 3 is a schematic illustration of a utilization of a coring tool, in accordance with some applications of the present invention;

FIGS. 4A-E are schematic illustrations of a utilization of apparatus for removing a lead, in accordance with some applications of the present invention;

FIG. 5 is a schematic illustration of a grasping tube, in accordance with some applications of the present invention;

FIGS. 6-7 are schematic illustrations of apparatus for removing a lead from a lumen of a subject, in accordance with some applications of the present invention;

FIG. 8 is a schematic illustration of a method for removing a lead, in accordance with some applications of the present invention; and

FIGS. 9A-D are schematic illustrations of a utilization of apparatus for removing a lead, in accordance with some applications of the present invention.

### DETAILED DESCRIPTION OF APPLICATIONS

Before referring to the figures, a general introduction to the present description is provided.

Applications of the present invention provide apparatus and methods for removing a lead (e.g., a cardiac pacemaker lead) from a lumen (e.g., a blood-vessel lumen) of a subject. Generally, as described hereinbelow, one or more substantially concentric tubes are advanced through the lumen toward a distal portion of the lead, such that, upon reaching the distal portion of the lead, substantially all of the lead is contained inside a lumen of the innermost tube. Typically, the lead is grasped multiple times as the tubes are advanced, thus facilitating advancement of the tubes in a controlled manner, and reducing undesired backward movement of the tubes. The lead is then grasped at the distal portion thereof, and removed from the lumen. Grasping the lead includes reducing a diameter of one or more grasping portions that are near the distal end of the innermost tube. The grasping portions are typically controlled from the proximal ends of the tubes, i.e., force and/or torque applied to the proximal end(s) is transferred to the grasping portions.

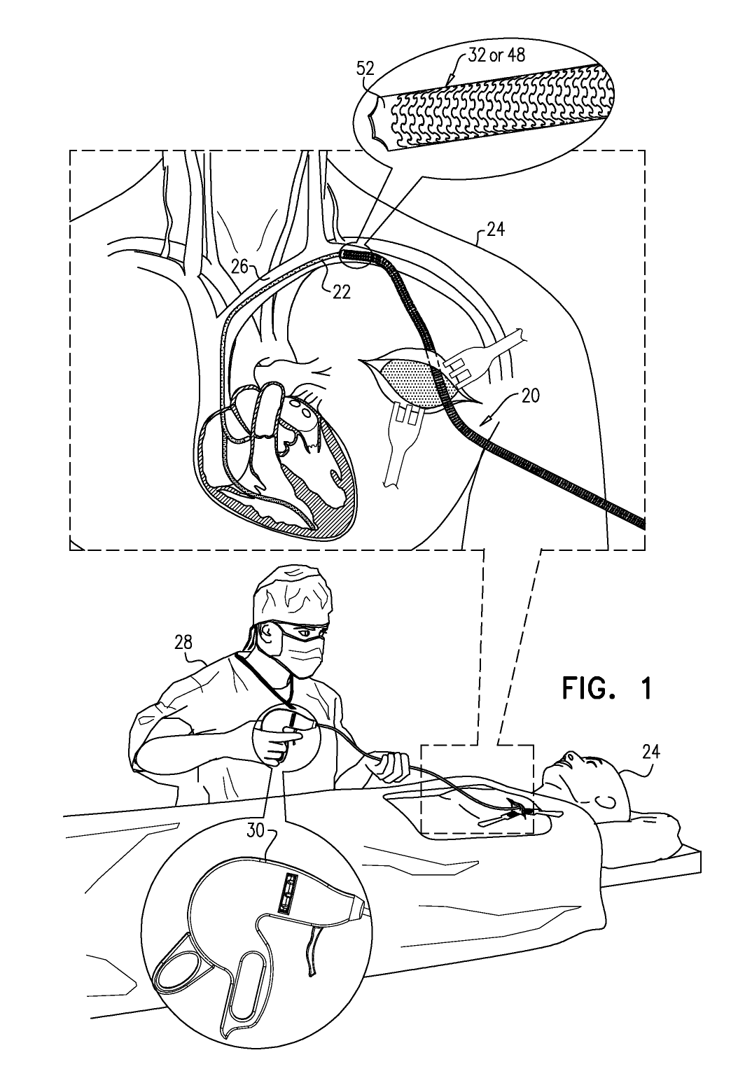

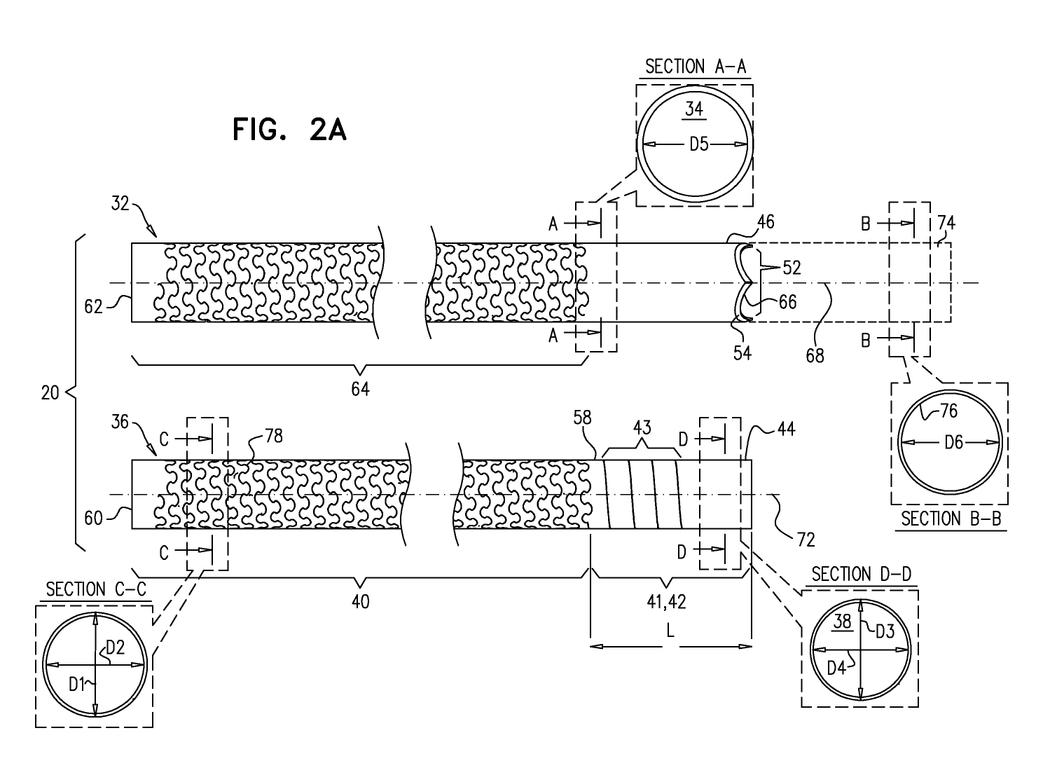

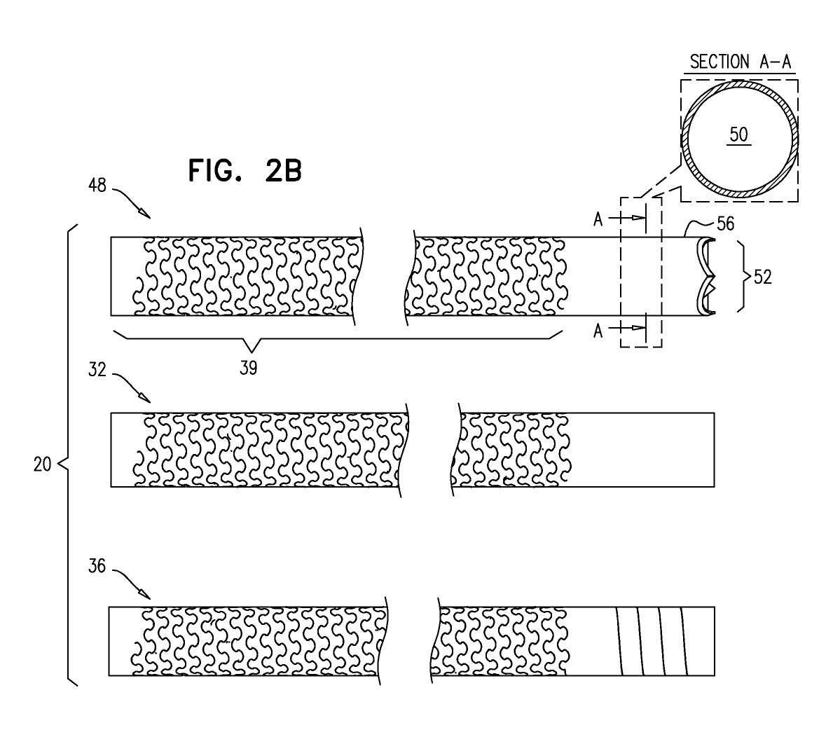

Reference is now made to FIG. 1, which is a schematic illustration of a method for removing a lead 22 from a lumen 26 of a subject 24, using apparatus 20, in accordance with some applications of the present invention. Reference is also made to FIGS. 2A-B, which are schematic illustrations of apparatus 20 in a disassembled state, in accordance with some applications of the present invention.

As shown in FIG. 2A, apparatus 20 comprises a flexible control tube 32 shaped to define a control-tube lumen 34. Apparatus 20 further comprises a flexible grasping tube 36, shaped to define a grasping-tube lumen 38. The grasping tube comprises a torque-transfer non-distal portion 40 and a distal portion 41 that is a helically-cut (e.g., via laser, water jet, or electric discharge machining (EDM)) distal portion 42. The grasping tube is disposed within control-tube lumen 34 such that helically-cut distal portion 42 is fixed (e.g., welded) at a distal end 44 thereof (which is also a distal end 44 of the grasping tube) to a distal end 46 of the control tube. (As noted above, FIGS. 2A-B show apparatus 20 in a disassembled state, such that grasping tube 36 is shown outside of control-tube lumen 34.) Typically, a coring tool 52, described hereinbelow, is disposed at distal end 46 of the control tube.

As shown in FIG. 1, apparatus 20 may be used to remove lead from lumen 26. The lead-removal method begins with the insertion of apparatus 20 into the lumen. Following the insertion of apparatus 20, distal portion 41 of the grasping tube, which includes a grasping portion 43, is advanced to a distal portion of the lead. (Helically-cut distal portion 42 is concealed inside of control tube 32.) The lead is then grasped within grasping portion 43, as described immediately below. Then, while grasping the lead within the grasping portion, the lead is removed. (It is noted that, with reference to the grasping of the lead and related functionality in the present description, the terms “distal portion” and “grasping portion” may be used interchangeably. For example, the lead may be alternatively described as being grasped within distal portion 41 or grasping portion 43.)

FIGS. 1 and 2A-B show applications in which distal portion 41 (i.e., helically-cut distal portion 42) is configured to grasp the lead within grasping-tube lumen 38 upon the proximal end 58 of distal portion 41 being rotated with respect to control tube 32. (Distal portion 41 is rotatable at least at proximal end 58, with respect to the control tube.) By virtue of distal end 44 of the grasping tube being fixed to distal end 46 of the control tube, distal end 44 is rotationally stationary with respect to the control tube; hence, the rotation of proximal end 58 with respect to the control tube reduces a diameter of grasping portion 43, such that the lead is grasped within the grasping portion. The rotation of proximal end 58 (and the reduction of the diameter of grasping portion 43) may be effected by rotating the proximal end 60 of the grasping tube with respect to the control tube, i.e., rotating proximal end 60 while not allowing proximal end 62 of the control tube to rotate in the same direction. Upon rotation of proximal end 60 of the grasping tube, torque-transfer portion 40 transfers the applied torque to proximal end 58 of distal portion 41, thus effecting the rotation of proximal end 58.

Alternatively or additionally, the reduction of the diameter of grasping portion 43 may be effected by rotating proximal end 62 of the control tube with respect to the proximal end of the grasping tube, i.e., rotating proximal end 62 while not allowing proximal end 60 of the grasping tube to rotate in the same direction. Upon rotation of proximal end 62, the non-distal torque-transfer portion 64 of the control tube, which is generally similar to non-distal torque-transfer portion 40 of the grasping tube, transfers the applied torque to distal end 46 of the control tube. Since the distal ends of the grasping tube and control tube are fixed to one another, the torque is transferred, in turn, to distal end 44 of the grasping tube, and the diameter of grasping portion 43 is reduced.

In some applications, the torque-transfer portion is laser-cut, the laser-cutting typically facilitating the transfer of torque, while preserving the flexibility of the grasping tube. For example, as shown in FIGS. 2A-B, the torque-transfer portion may comprise a self-interlocking helical ribbon 78 having a circumference that does not change upon the ribbon being rotated with respect to the control tube. In other applications (not shown), the torque transfer portion is a stranded wire with an open center working channel, such as the Helical Hollow Strand™ Tube by Fort Wayne Metals.

As shown in FIG. 1, a physician 28 may control the advancement of the apparatus, the grasping of the lead, and the removal of the lead, using a control handle 30.

Typically, grasping tube 36 is cylindrical, and an outer diameter D1 and/or an inner diameter D2 of the torque-transfer non-distal portion of the grasping tube is at least 2 mm and/or less than 6 mm. Further typically, an outer diameter D3 and/or an inner diameter D4 of the helically-cut distal portion, in the absence of any external force or torque applied to the helically-cut distal portion, is at least 2 mm and/or less than 6 mm. Typically, control tube 32 is also cylindrical, and an inner diameter D5 of the control tube is at least 0.01 mm and/or less than 0.1 mm larger than outer diameter D1 and/or outer diameter D3 of the grasping tube. Typically, a length L of the helically-cut distal portion along a longitudinal axis 72 of the helically-cut distal portion, in the absence of any external force or torque applied to the helically-cut distal portion, is at least 5 mm and/or less than 30 mm.

Typically, coring tool 52 comprises one or more (e.g., a plurality of) blades 54, shaped to define respective cutting edges 66. Typically, blades 54 are disposed around a central longitudinal axis 68 of the control tube. Cutting edges 66 typically face toward central longitudinal axis 68 of the control tube, this orientation typically facilitating safe and effective separation of the lead from tissue of the subject. (For example, this orientation may help prevent the cutting edges from cutting tissue of the subject upon withdrawal of the apparatus from the subject.) Typically, the cutting edges are disposed such that the tissue is cut relatively close to the lead. For example, cutting edges 66 may be within 0.1 and 0.3 mm of an inside wall 76 of a hypothetical cylinder 74 having an inner diameter D6 equal to D5 and extending from the distal end of the control tube to a point that is distal to the coring tool.

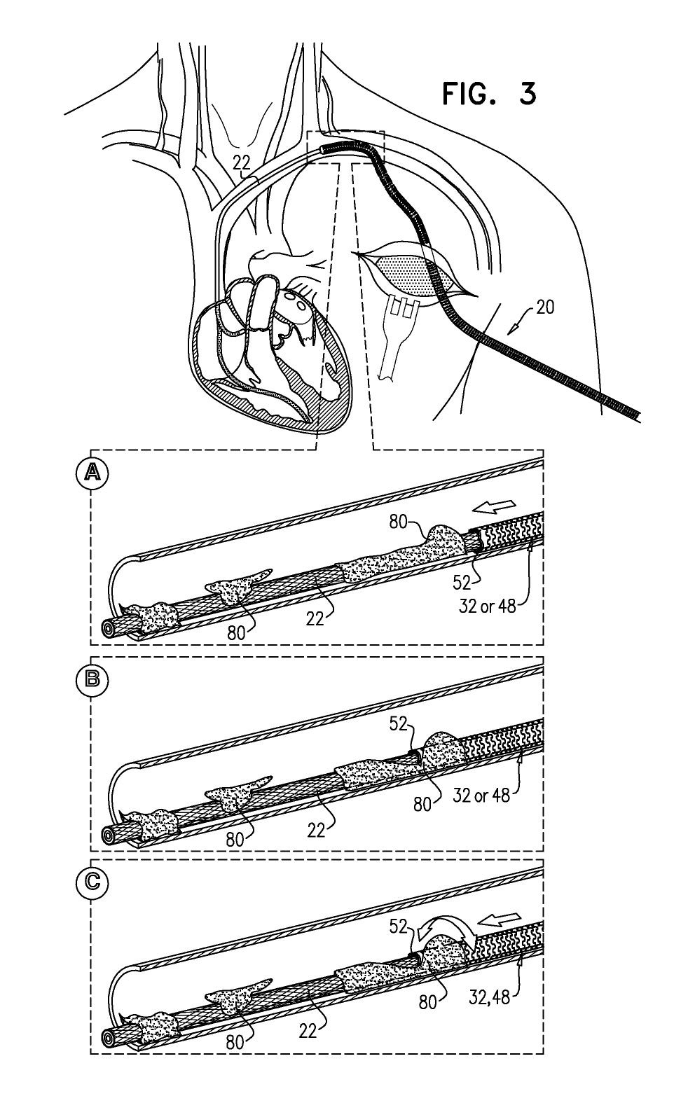

In some applications, as shown in FIG. 2B, apparatus 20 further comprises a flexible outer tube 48 shaped to define an outer-tube lumen 50, and control tube 32 is a middle tube shaped to fit inside the outer-tube lumen. In such applications, coring tool 52 is typically disposed at a distal end 56 of outer tube 48. In general, coring tool 52, when disposed at the end of outer tube 48, has some or all of the properties that were described above with reference to FIG. 2A, mutatis mutandis. For example, cutting edges 66 typically face toward a central longitudinal axis of the outer tube. Typically, outer tube 48 comprises an outer-tube torque-transfer non-distal portion 39, which is generally similar to torque-transfer non-distal portion 40 of the grasping tube. Outer-tube torque-transfer non-distal portion 39 facilitates rotation of the outer tube about its longitudinal axis, e.g., for coring tissue or releasing cored tissue from the outer tube, as described hereinbelow with reference to FIG. 3.

In some applications, (a) the outer tube, and/or (b) the control tube or grasping tube, does not comprise a torque-transfer non-distal portion. (Typically, a tube that does not comprise a torque-transfer non-distal portion is not rotated about its longitudinal axis, during the removal of the lead.)

It is noted that FIGS. 1 and 3 may be said to alternatively depict control tube 32 or outer tube 48 as the outermost tube of the apparatus, per the respective applications shown in FIGS. 2A-B. For simplicity, however, and without loss of generality, the description of FIG. 1 above relates exclusively to control tube 32 as the outermost tube of the apparatus, while the description of FIG. 3 below relates exclusively to outer tube 48 as the outermost tube of the apparatus.

Reference is now made to FIG. 3, which is a schematic illustration of a utilization of coring tool 52, in accordance with some applications of the present invention. FIG. 3 shows coring tool 52 being used to separate lead 22 from tissue 80 of the subject by coring the tissue, while distal portion 41 of the grasping tube (which is concealed) is advanced to the distal portion of the lead. Typically, the coring is performed by advancing outer tube 48 a relatively small distance forward (sub-figure A), such that coring tool 52, which is disposed at the distal end 56 of outer tube 48, cores a small amount of tissue 80, thus separating the tissue from the lead (sub-figure B). In some applications, outer tube 48 is rotated about its longitudinal axis while being advanced (sub-figure C), or in alternation with the advancement of outer tube 48. The rotation of outer tube 48 may facilitate the coring of tissue, and/or the release of cored tissue.

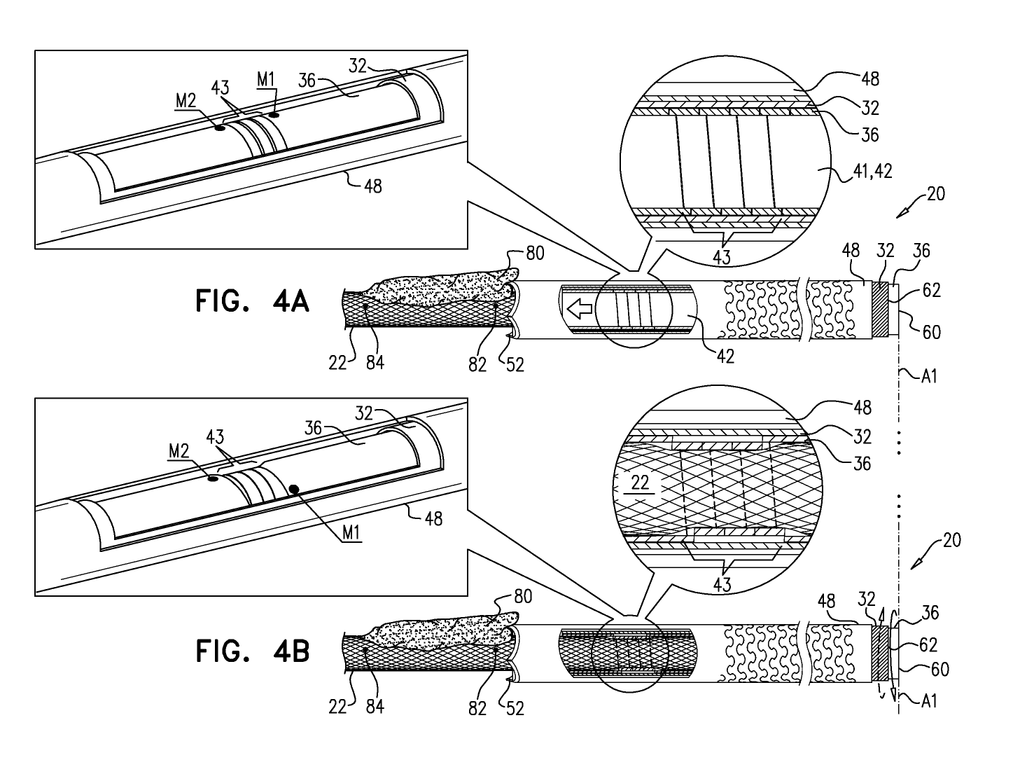

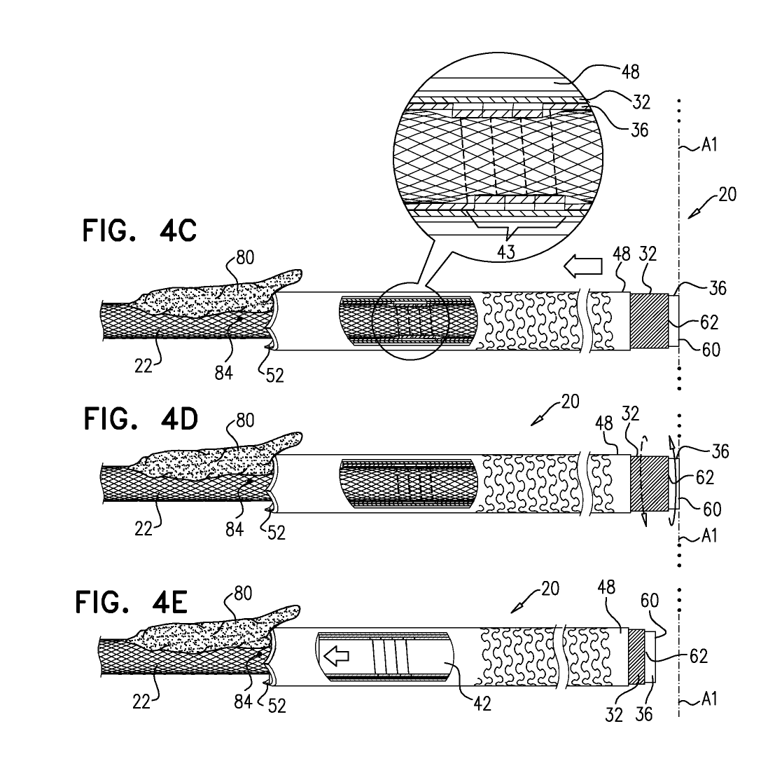

Reference is now made to FIGS. 4A-E, which are schematic illustrations of a utilization of apparatus 20, in accordance with some applications of the present invention. In some applications, the advancement of distal portion 41 of grasping tube 36 to the distal portion of lead 22 comprises repeatedly executing a sequence of steps, at least some of these steps being shown in FIGS. 4A-E, as follows:

(i) Distal portion 41 of grasping tube 36 is advanced to a site adjacent to a first tissue-to-lead adhesion site 82, as shown in FIG. 4A. Typically, distal portion 41 is advanced by advancing proximal end 60 of the grasping tube. Position-marking line A1 marks the position of proximal end 60 following the advancement thereof.

(ii) At the site adjacent to tissue-to-lead adhesion site 82, lead 22 is grasped within grasping portion 43, as shown in FIG. 4B. Lead 22 is grasped by reducing the diameter of the grasping portion, e.g., by rotating proximal end 60 of grasping tube 36, as described hereinabove and as depicted in FIG. 4B by the solid arrow. (Alternatively or additionally, the diameter of the grasping portion may be reduced by rotating proximal end 62 of control tube 32 in the opposite direction, as described hereinabove and as depicted in FIG. 4B by the dashed arrow.) In the example shown in FIG. 4B, proximal end 60 is rotated in a counterclockwise direction (viewed from the proximal end), such that the proximal side of grasping portion 43 rotates in a counterclockwise direction, relative to the (generally stationary) distal side of grasping portion 43. This is depicted in FIGS. 4A-B by the movement of a proximal marker M1 relative to a distal marker M2. (Markers M1 and M2 are not part of the apparatus, but rather, are included in FIGS. 4A-B for illustrative purposes only.) For applications in which proximal end 62 of the control tube is rotated alternatively or additionally to the rotation of proximal end 60 of the grasping tube, the distal side of grasping portion 43 rotates relative to the proximal side of the grasping portion. For example, if the proximal ends of both tubes were to be rotated as depicted by the solid arrow and dashed arrow in FIG. 4B, distal marker M2 would rotate clockwise relative to proximal marker M1, while marker M1 would rotate counterclockwise relative to marker M2.

(iii) While grasping the lead within the grasping portion, coring tool 52 is used to separate the lead from tissue 80 at tissue-to-lead adhesion site 82, as shown in FIG. 4C and described hereinabove with reference to FIG. 3.

(iv) As shown in FIG. 4D, the lead is released from grasping portion 43, e.g., by rotating proximal end 60 of the grasping tube and/or proximal end 62 of the control tube in the rotational direction(s) opposite from the rotational direction(s) of FIGS. 2A-B.

(v) Following the separating of the lead from the tissue at the first tissue-to-lead adhesion site (and the release of the lead from the grasping portion), the distal portion of the grasping tube is advanced to a site adjacent to a second tissue-to-lead adhesion site 84, as shown in FIG. 4E. Typically, the distal portion of the grasping tube is advanced by advancing the proximal end of the grasping tube to a position distal to position-marking line A1.

Reference is now made to FIG. 5, which is a schematic illustration of grasping tube 36, in accordance with some applications of the present invention. In some applications, grasping tube 36 comprises a first helically-cut distal portion 42a (including a first grasping portion 43a), and a second helically-cut distal portion 42b (including a second grasping portion 43b) that is proximal thereto. The two helically-cut distal portions are separated from each other by a portion of the grasping tube that is not cut.

As shown, the handedness of one of the helically-cut distal portions is different from the handedness of the other, i.e., one of the helically-cut distal portions is right-handed, while the other is left-handed. Thus, (a) first helically-cut distal portion 42a is configured to grasp the lead within the grasping-tube lumen upon a proximal end 58a thereof being rotated with respect to the control tube in a first direction, while (b) second helically-cut distal portion 42b is configured to grasp the lead within the grasping-tube lumen upon a proximal end thereof being rotated with respect to the control tube in a second direction that is opposite from the first direction. For example, in the application shown in FIG. 5, first helically-cut distal portion 42a grasps the lead upon proximal end 58a being rotated in a clockwise direction (viewed from the proximal end of the grasping tube), while second helically-cut distal portion 42b grasps the lead upon proximal end 58b being rotated in a counterclockwise direction.

As described hereinabove with reference to FIGS. 2A-B, proximal ends 58a and 58b may be rotated by rotating the proximal end of the grasping tube and/or the proximal end of the control tube. For example, rotating the proximal end of the grasping tube in a clockwise direction may result in a reduction in the diameter of first grasping portion 43a and the grasping of the lead by first helically-cut distal portion 42a. Similarly, rotating the proximal end of the grasping tube in the opposite (counterclockwise) direction may result in a reduction in the diameter of second grasping portion 43b and the grasping of the lead by second helically-cut distal portion 42b.

Typically, the application shown in FIG. 5 is used to advance the distal portion of grasping tube 36 to the distal portion of the lead by repeatedly executing a sequence of steps that includes:

(a) grasping the lead within first grasping portion 43a, by reducing a diameter of the first grasping portion, e.g., by rotating proximal end 60 of the grasping tube;

(b) while grasping the lead within first grasping portion 43a, advancing proximal end 58b of the second grasping portion;

(c) following the advancing of proximal end 58b, grasping the lead within second grasping portion 43b, by reducing a diameter of the second grasping portion, e.g., by rotating proximal end 60; and

(d) while grasping the lead within second grasping portion 43b, advancing distal end 44 of the grasping tube.

Repeated execution of the above sequence of steps will typically cause grasping tube 36 to “creep” forward in a caterpillar-like fashion.

The two-helix application of apparatus 20 shown in FIG. 5, and the advancement of distal portion 41 in the creeping manner described above, are typically used and practiced in combination with at least some aspects of the method shown in FIGS. 4A-E. For example, the following sequence of steps may be executed:

(i) While grasping the lead within second grasping portion 43b (similarly to what is shown in FIG. 4B), coring tool 52 is used to separate the lead from tissue 80 at tissue-to-lead adhesion site 82 (FIG. 4C).

(ii) The lead is released from second grasping portion 43b (similarly to what is shown FIG. 4D), and subsequently, grasped by first grasping portion 43a (per step (a) above).

(iii) While grasping the lead within first grasping portion 43a, proximal end 58b of second grasping portion 43b is advanced (per step (b) above).

(iv) Following the advancing of proximal end 58b, the lead is released from first grasping portion 43a, and subsequently, grasped within second grasping portion 43b (per step (c) above).

(v) While grasping the lead within second grasping portion 43b, distal end 44 of the grasping tube is advanced (per step (d) above, and as shown in FIG. 4E).

Since the lead is being grasped substantially the entire time by at least one of the two grasping portions, undesired backward sliding of grasping tube 36 is reduced.

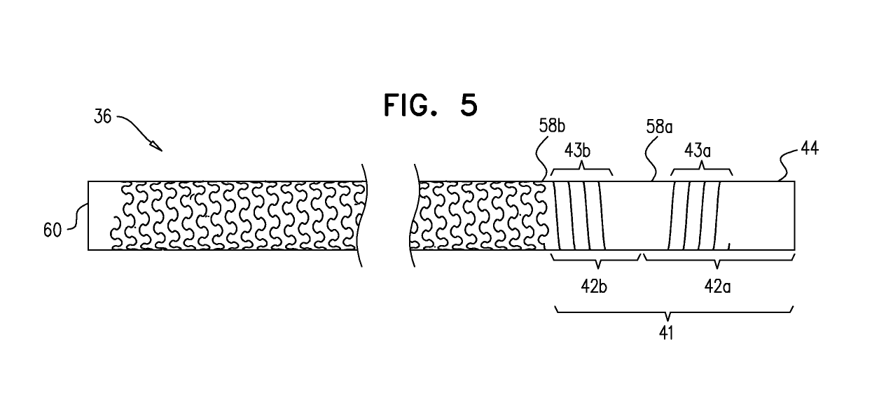

Reference is now made to FIG. 6, which is a schematic illustration of apparatus 21 for removing a lead 22 (e.g., a cardiac pacemaker lead) from a lumen 26 (e.g., a blood-vessel lumen) of a subject 24, in accordance with some applications of the present invention. Apparatus 21 is conceptually similar to apparatus 20, but differs from apparatus 20 in several ways, particularly with respect to the manner in which the apparatus grasps the lead.

Like apparatus 20, apparatus 21 comprises flexible grasping tube 36, shaped to define a tube lumen, i.e., grasping-tube lumen 38. However, in apparatus 21, distal portion 41 of the grasping tube is not helically-cut; rather, distal portion 41 comprises a distal braided portion 86 configured to grasp the lead within the tube lumen upon being longitudinally stretched. (Since distal braided portion 86 functions as a grasping portion 43, the terms “distal braided portion” and “grasping portion” may be used interchangeably in some contexts of the present description of apparatus 21. For example, either distal braided portion 86 or grasping portion 43 may be said to grasp the lead.) Distal braided portion 86 is controlled via longitudinal control elements 88, which comprise a first longitudinal control element 90 and a second longitudinal control element 92. First longitudinal control element 90 extends from a proximal end of apparatus 21 (not shown) to a distal end 94 of the distal braided portion, while second longitudinal control element 92 extends from the proximal end of the apparatus to a proximal end 96 of the distal braided portion.

Longitudinal control elements 88 comprise respective flexible tubes 98, shaped to define respective flexible-tube lumens 100, and respective longitudinal elements 102 shaped to fit inside flexible-tube lumens 100. Longitudinal elements 102 transmit both compressive and tensile force from the proximal ends of the longitudinal control elements to the distal ends thereof, thus facilitating the control of distal braided portion 86. Typically, longitudinal elements 102 are wires having respective diameters D7/D7′ between 0.1 and 1 mm. Also typically, respective inner diameters D8/D8′ of flexible tubes 98 are between 0.2 and 2 mm greater than respective diameters D7/D7′.

Typically, apparatus 21 comprises coring tool 52, which, as described above, comprises one or more blades 54. In some applications, coring tool 54 is disposed at distal end 44 of the grasping tube. In other applications, as shown in FIG. 2A with respect to apparatus 20, apparatus 21 further comprises control tube 32, grasping tube 36 is shaped to fit inside control-tube lumen 34, and coring tool 52 is disposed at distal end 46 of the control tube.

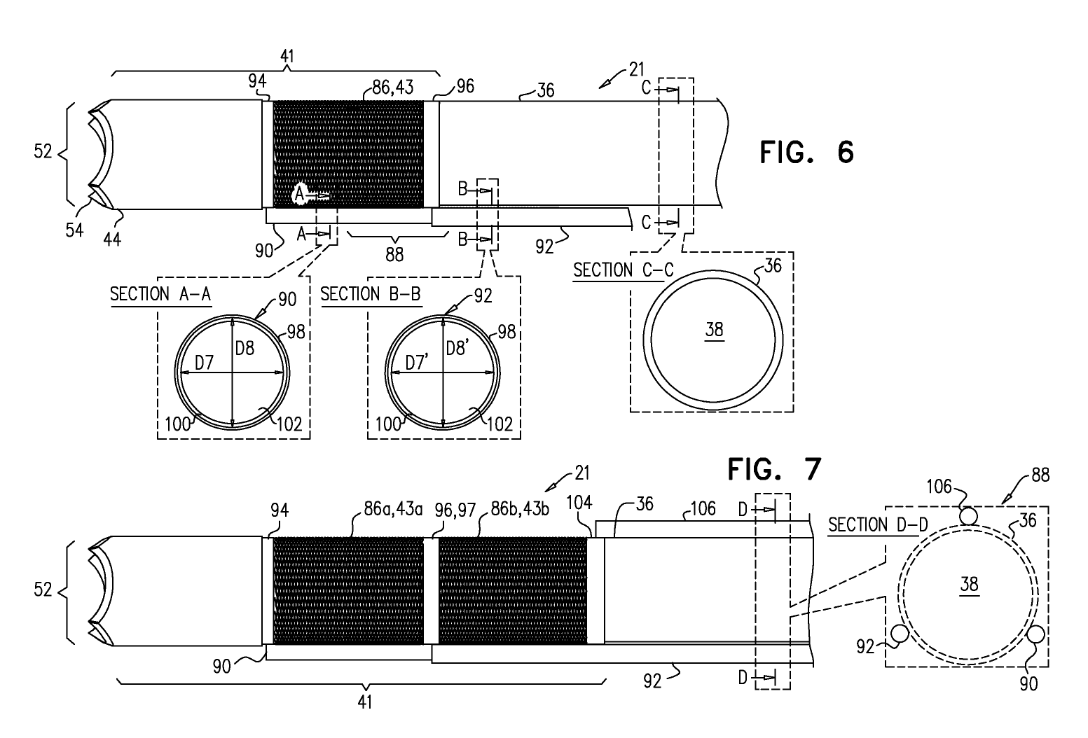

Reference is now made to FIG. 7, which is a schematic illustration of apparatus 21, in accordance with some applications of the present invention. In some applications, grasping tube 36 comprises two distal braided portions: a first distal braided portion 86a (i.e., a first grasping portion 43a), and a second distal braided portion 86b (i.e., a second grasping portion 43b). As shown in FIG. 7, second distal braided portion 86b is proximal to first distal braided portion 86a, and is separated from the first distal braided portion by a non-braided portion 97 of the grasping tube, e.g., proximal end 96 of the first distal braided portion. As described above with reference to FIG. 6, each distal braided portion is configured to grasp the lead within tube lumen upon being longitudinally stretched. The distal braided portions are controlled via longitudinal control elements 88, which, in the application shown in FIG. 7, further comprise a third longitudinal control element 106 extending from the proximal end of the apparatus to a proximal end 104 of the second distal braided portion. (Typically, the properties of third longitudinal control element 106 are substantially the same as those of the first and second longitudinal control elements.)

The cross-section of FIG. 7 is a view of apparatus 21 from the proximal end thereof. As shown, first longitudinal control element 90 is separated from second longitudinal control element by approximately 90-120 degrees, measured along the circumference of the grasping tube, such that it is mostly concealed behind the second longitudinal control element in the main side-view of FIG. 7. This configuration is only one of many possible configurations of longitudinal control elements 88 that are included in the scope of the present invention.

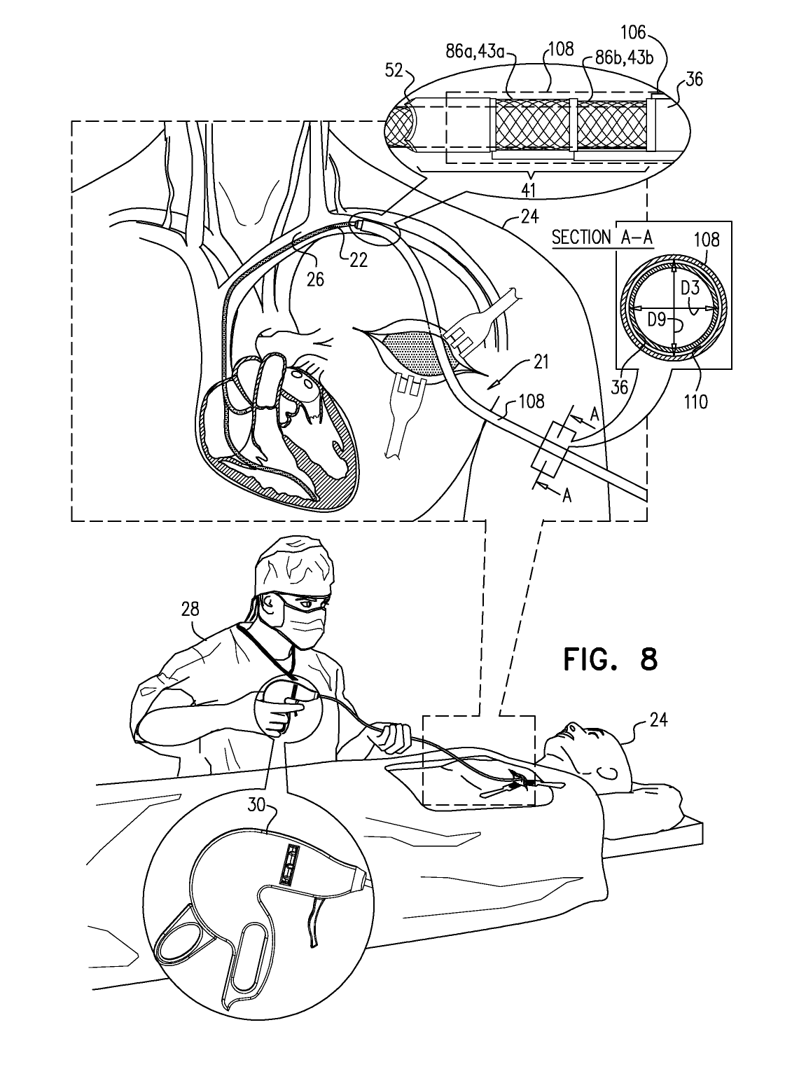

Reference is now made to FIG. 8, which is a schematic illustration of a method for removing lead 22 using apparatus 21, in accordance with some applications of the present invention. The method for removal of lead 22 that is depicted in FIG. 8 is generally similar to the method depicted in FIG. 1. First, apparatus 21 is inserted into lumen 26. Following the insertion of apparatus 21, distal portion 41 of the grasping tube, which includes one distal braided portion 86 (i.e., one grasping portion 43) or, alternatively, two distal braided portions 86a and 86b (i.e., two grasping portions 43a and 43b), is advanced to a distal portion of the lead. The lead is then grasped within grasping portion 43, or within one or both of grasping portions 43a and 43b, by reducing a diameter of the grasping portion(s). Then, while grasping the lead within the grasping portion(s), the lead is removed.

As shown in FIG. 8, apparatus 21 typically comprises a catheter 108 (e.g., a steerable catheter) shaped to define a catheter lumen 110, and grasping tube 36 is shaped to fit inside catheter lumen 110. Typically, an inner diameter D9 of catheter 108 is between 0.05 and 1 mm greater than outer diameter D3 of the grasping tube. Grasping tube 36 is generally inserted into lumen 26 while inside of catheter 108. Furthermore, during most of the advancement of grasping tube 36 through lumen 26, grasping tube 36 is typically covered by catheter 108, with the distal end of the grasping tube emerging from the distal end of the catheter only on occasion (e.g., for coring of tissue). Catheter 108 generally helps to protect the inside of the blood vessel from being damaged by apparatus 21. In some applications, catheter 108 may be used in combination with apparatus 20 (FIG. 1), in a generally similar manner.

Reference is again made to FIG. 6. To reduce the diameter of grasping portion 43 (and thus grasp the lead), the grasping portion is longitudinally stretched. Typically, this stretching is effected by pulling second longitudinal control element 92 and/or pushing longitudinal control element 90. Similarly, with reference to FIG. 7, reducing the diameter of second grasping portion 43b comprises pushing second longitudinal control element 92 and/or pulling third longitudinal control element 106, while reducing the diameter of first grasping portion 43a comprises pushing first longitudinal control element 90 and/or pulling second longitudinal control element 92.

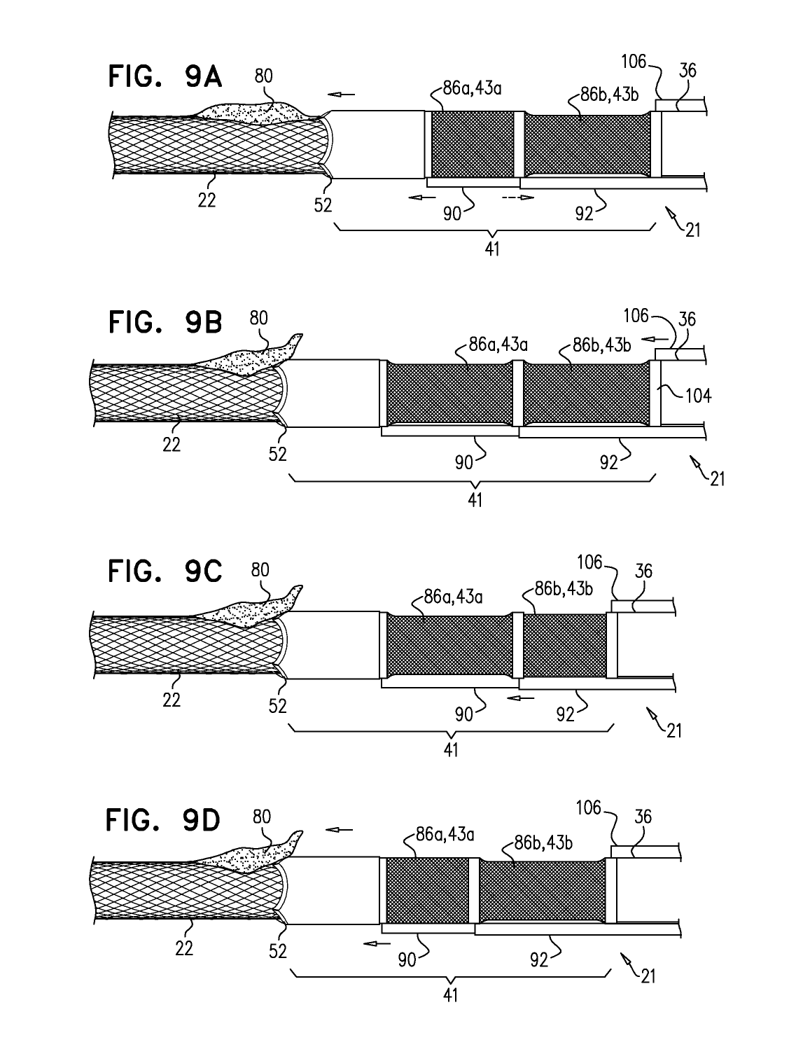

Reference is now made to FIGS. 9A-D, which are schematic illustrations of a utilization of apparatus 21, in accordance with some applications of the present invention. FIGS. 9A-D show a typical sequence of steps that is repeatedly executed while advancing distal portion 41 of grasping tube 36 to the distal portion of lead 22. FIGS. 9A-D show the application of FIG. 7, in which the distal portion of the grasping tube includes two grasping portions. (Each of the small open-headed arrows of FIGS. 9A-D, as opposed to the large open-bodied arrows of FIG. 4A and FIG. 4E, depicts the performance of an action the result of which is shown in the figure that follows the figure containing the small open-headed arrow. For example, the result of an action depicted by a small open-headed arrow in FIG. 9A is shown in FIG. 9B, not in FIG. 9A.) These steps are as follows:

(i) As depicted by the arrows in FIG. 9A, the lead is grasped within first grasping portion 43a, by reducing a diameter of the first grasping portion. Typically, the diameter of the first grasping portion is reduced by pushing first longitudinal control element 90 while inhibiting second longitudinal control element 92 from moving forward. (Thus, the distal end of apparatus 21 is advanced, and coring tool 52 may core tissue 80 of the subject.) Alternatively or additionally, as depicted by the dashed arrow below the distal end of second longitudinal control element 92, the diameter of the first grasping portion is reduced by pulling second longitudinal control element 92 while inhibiting first longitudinal control element 90 from moving back. (Typically, before and during the grasping of the lead within first grasping portion 43a, the lead continues to be grasped within second grasping portion 43b, as shown in FIG. 9A.) As a result of pushing first longitudinal control element 90, first grasping portion 43a is elongated, as shown in FIG. 9B.

(ii) While grasping the lead within the first grasping portion, proximal end 104 of the second grasping portion is advanced, typically by pushing third longitudinal control element 106 while inhibiting second longitudinal control element 92 from moving forward, as depicted by the arrow in FIG. 9B. As a result, the length of second grasping portion 43b is reduced to its resting length (i.e., its length when no external force is applied to it), as shown in FIG. 9C. (Alternatively, the length of second grasping portion 43b may be reduced to a length that is less than its resting length.)

(iii) Next, as shown in FIG. 9C, the lead is grasped within second grasping portion 43b, by reducing a diameter of the second grasping portion. Typically, the diameter of the second grasping portion is reduced by pushing second longitudinal control element 92 while inhibiting third longitudinal control element 106 from moving forward. As a result, the second grasping portion is elongated, and the length of the first grasping portion is reduced to its resting length, as shown in FIG. 9D. (Alternatively, the length of the first grasping portion may be reduced to a length that is less than its resting length.)

(iv) Next, while grasping the lead within the second grasping portion, distal end 41 of the grasping tube is advanced, typically by pushing first longitudinal control element 90. (This step may include coring of tissue 80 by coring tool 52.) Typically, as shown in FIG. 9D, second longitudinal control element 92 is inhibited from moving forward, such that the lead is grasped in the first grasping portion. (In such applications, steps (i) and (iv) may occur concurrently, and FIG. 9D may be said to depict the same operation that is depicted in FIG. 9A.)

Repeated execution of the above sequence of steps will typically cause grasping tube 36 to “creep” forward in a caterpillar-like fashion. Since the lead is being grasped substantially the entire time by at least one of the two grasping portions, undesired backward sliding of grasping tube 36 is reduced.

In general, elements of FIGS. 1-5 and the description thereof may be practiced in combination with elements of FIGS. 6-9 and the description thereof. For example, the utilization of coring tool 52 that was described hereinabove with reference to FIG. 3 may be practiced in combination with steps (i) and/or (iv) described hereinabove with reference to FIGS. 9A-D.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for removing a lead from a lumen of a subject, the apparatus comprising:
a flexible control tube shaped to define a control-tube lumen; and
a flexible grasping tube shaped to define a grasping-tube lumen, the grasping tube being disposed within the control-tube lumen and comprising:
a torque-transfer non-distal portion; and
a helically-cut distal portion, that is (a) fixed at a distal end thereof to a distal end of the control tube, and (b) rotatable at least at a proximal end thereof with respect to the control tube,
the helically-cut distal portion being configured to grasp the lead within the grasping-tube lumen upon the proximal end of the helically-cut distal portion being rotated with respect to the control tube.

2. The apparatus according to claim 1, wherein the grasping tube is cylindrical, and wherein a diameter of the torque-transfer non-distal portion of the grasping tube is between 2 and 6 mm.

3. The apparatus according to claim 1, wherein the control tube is cylindrical, and wherein an inner diameter of the control tube is between 0.01 and 0.1 mm larger than an outer diameter of the grasping tube.

4. The apparatus according to claim 1, wherein a length of the helically-cut distal portion along a longitudinal axis of the helically-cut distal portion, in the absence of any external force or torque applied to the helically-cut distal portion, is between 5 and 30 mm.

5. The apparatus according to claim 1, wherein the grasping tube is cylindrical, and wherein a diameter of the helically-cut distal portion, in the absence of any external force or torque applied to the helically-cut distal portion, is between 2 and 6 mm.

6. The apparatus according to claim 1, further comprising, at a distal end of the control tube, a coring tool comprising one or more blades shaped to define respective cutting edges.

7. The apparatus according to claim 6, wherein the cutting edges face toward a central longitudinal axis of the control tube.

8. The apparatus according to claim 6, wherein the one or more blades comprise a plurality of blades disposed around a central longitudinal axis of the control tube.

9. The apparatus according to claim 6, wherein the control tube has a control tube inner diameter, and wherein the cutting edges are within 0.1 and 0.3 mm of an inside wall of a hypothetical cylinder having an inner diameter equal to the control tube inner diameter and extending from the distal end of the control tube to a point that is distal to the coring tool.

10. The apparatus according to claim 1, further comprising:
a flexible outer tube shaped to define an outer-tube lumen, the control tube being a middle tube shaped to fit inside the outer-tube lumen; and
a coring tool comprising one or more blades and disposed at a distal end of the outer tube.

11. The apparatus according to claim 1, wherein the torque-transfer non-distal portion of the grasping tube comprises a laser-cut torque-transfer non-distal portion of the grasping tube.

12. The apparatus according to claim 11, wherein the laser-cut torque-transfer non-distal portion of the grasping tube comprises a self-interlocking helical ribbon having a circumference that does not change upon the ribbon being rotated with respect to the control tube.

13. The apparatus according to claim 1,
wherein the helically-cut distal portion of the grasping tube is a first helically-cut distal portion of the grasping tube that is configured to grasp the lead within the grasping-tube lumen upon a proximal end of the first helically-cut distal portion being rotated with respect to the control tube in a first direction, and
wherein the grasping tube further comprises a second helically-cut distal portion that is proximal to the first helically-cut distal portion, the second helically-cut distal portion being configured to grasp the lead within the grasping-tube lumen upon a proximal end of the second helically-cut distal portion being rotated with respect to the control tube in a second direction that is opposite from the first direction.

14-36. (canceled)
